(19) United States
(12) Patent Application Publication
(10) Pub. No.: US 2026/0165871 A1
(43) Pub. Date: Jun. 18, 2026

(54) FLUID COLLECTION ASSEMBLIES INCLUDING A HYDROPHILIC FLUID PERMEABLE OUTER LAYER

(71) Applicant: PUREWICK CORPORATION, Covington, GA (US)

(72) Inventors: Zhihui Yin, Lilburn, GA (US); Michael Anderson, Monroe, GA (US)

(21) Appl. No.: 19/127,234

(22) PCT Filed: Nov. 8, 2022
(86) PCT No.: PCT/US2022/049300

§ 371 Date: May 5, 2025

(51) Int. Cl. A61F 5/44; A61F 5/455; A61L 15/24; A61L 15/28; A61L 15/42; A61M 1/00

(52) U.S. Cl. CPC A61F 5/4401; A61F 5/455; A61L 15/24; A61L 15/28; A61L 15/425; A61M 1/87; A61M 2202/0496

(57) ABSTRACT

An example fluid collection assembly includes a fluid impermeable layer. The fluid impermeable layer at least defines a chamber, at least one opening that allows bodily fluids to enter the chamber, and a fluid outlet that allows the bodily fluids to be removed from the chamber. The fluid collection assembly also include a porous material at least partially disposed in the chamber. The porous material includes a hydrophilic fluid permeable outer layer and a fluid permeable inner layer. The fluid permeable inner layer includes at least one of a foam or a non-woven material.

### BACKGROUND

A person or animal may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden individuals are sometimes used. However, bedpans can be prone to discomfort, spills, and other hygiene issues.

### SUMMARY

Embodiments are directed to fluid collection assemblies, fluid collection systems including the same, and methods of making and using the same. An example fluid collection assembly includes a fluid impermeable layer (e.g., a fluid impermeable barrier). In an embodiment, a fluid collection assembly is disclosed. The fluid collection assembly includes a fluid impermeable layer defining at least a chamber, at least one opening, and a fluid outlet. The fluid collection assembly also includes a porous material at least partially disposed in the chamber. The porous material includes a hydrophilic fluid permeable outer layer and a fluid permeable inner layer including at least one of a non-woven material or a foam.

In an embodiment, a fluid collection system is disclosed. The fluid collection system includes a fluid collection assembly. The fluid collection assembly includes a fluid impermeable layer defining at least a chamber, at least one opening, and a fluid outlet. The fluid collection assembly also includes a porous material at least partially disposed in the chamber. The porous material includes a hydrophilic fluid permeable outer layer and a fluid permeable inner layer including at least one of a non-woven material or a foam. The fluid collection system also includes a fluid storage container and a vacuum source. The chamber of the fluid collection assembly, the fluid storage container, and the vacuum source are in fluid communication with each other that, when one or more bodily fluids are present in the chamber, a suction provided from the vacuum source to the chamber of the fluid collection assembly removes the one or more bodily fluids from the chamber and deposits the bodily fluids in the fluid storage container.

In an embodiment, a method to collect bodily fluids is disclosed. The method includes positioning at least one opening of a fluid collection assembly adjacent to a female urethral opening. The fluid collection assembly includes a fluid impermeable layer defining at least a chamber, the at least one opening, and a fluid outlet. The fluid collection assembly also includes a porous material at least partially disposed in the chamber. The porous material includes a hydrophilic fluid permeable outer layer and a fluid permeable inner layer including at least one of a foam or a non-woven material. The method also includes receiving bodily fluids from the female urethral opening into the chamber.

In an embodiment, a method to form a fluid collection assembly is disclosed. The method includes providing a porous material. The porous material includes a hydrophilic fluid permeable outer layer and a fluid permeable inner layer. The method also includes disposing the porous material through at least one opening and into a chamber. The fluid permeable inner layer includes at least one of a nonwoven material or a foam. The at least one opening and the chamber is defined by a fluid impermeable layer. The fluid impermeable layer also defines a fluid inlet.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly, according to an embodiment.

FIGS. 1B and 1C are cross-sectional schematics of the fluid collection assembly taken along planes 1B-1B and 1C-1C, respectively, according to an embodiment.

FIG. 2 is a schematic illustration of a method to form the porous material, according to an embodiment.

FIG. 3A is an isometric view of a fluid collection assembly, according to an embodiment.

FIGS. 3B and 3C are cross-sectional schematics of the fluid collection assembly taken along planes 3B-3B and 3C-3C, respectively, according to an embodiment.

FIG. 4 is a block diagram of a fluid collection system for fluid collection, according to an embodiment.

### DETAILED DESCRIPTION

Embodiments are directed to fluid collection assemblies, fluid collection systems including the same, and methods of making and using the same. An example fluid collection assembly includes a fluid impermeable layer (e.g., a fluid impermeable barrier). The fluid impermeable layer at least defines a chamber, at least one opening that allows bodily fluids (e.g., urine, blood, sweat, etc.) to enter the chamber, and a fluid outlet that allows the bodily fluids to be removed from the chamber. The fluid collection assembly also includes a porous material at least partially disposed in the chamber. The porous material includes a hydrophilic fluid permeable outer layer (“outer layer”) and a fluid permeable inner layer (“inner layer”). The fluid permeable inner layer includes a foam or a non-woven material.

During use, the fluid collection assembly may be positioned such that the opening and the portions of the porous material extending across the opening are positioned adjacent to a female urethral opening. After positioning the fluid collection assembly, the individual may discharge bodily fluids from the urethral opening. The discharged bodily fluids may flow through the opening and into chamber. For example, the bodily fluids may be received into the outer layer and flow into the inner layer. The bodily fluids may then be removed from the chamber via the fluid outlet, such as using a conduit that is positioned through the fluid outlet. In a particular example, a suction may be provide to the chamber via a vacuum source that is in fluid communication with the chamber (e.g., via the conduit). The suction may cause the bodily fluids in the porous material to flow generally towards the fluid outlet and/or an inlet of a conduit that is positioned through the fluid outlet. The suction then removes the bodily fluids that reach the fluid outlet and/or the inlet of the conduit. Bodily fluids removed from the chamber may be deposited in a fluid storage container that is in fluid communication with both the chamber and the vacuum source.

The porous materials of some conventional fluid collection assemblies include a gauze, cross-lapped porous nonwoven materials, or other porous materials that are positioned to initially receive bodily fluids from the individual using such conventional fluid collection assemblies. Such porous materials are configured to be hydrophobic. However, it has been found that many of the gauzes, cross-lapped nonwoven materials, and other porous materials positioned to initially receive the bodily fluids from the individual may be inefficient at capturing the bodily fluids discharged from the individual which increases the likelihood that bodily fluids leak from the conventional fluid collection assemblies. Further, it has been found that many of the gauzes and other porous materials remain wet after the individual discharges bodily fluids which prevent the conventional fluid collection assemblies from being used for a prolonged period of time (e.g., periods of time greater than 12 hours) without causing skin degradation of the individual.

The porous materials of the fluid collection assemblies disclosed herein (i.e., the porous materials that include an outer layer and an inner layer) remedy at least some of these issues associated with the porous materials of conventional fluid collection assemblies. For example, the outer layer is configured to efficiently receive the bodily fluids, thereby preventing or at least inhibiting leakage of the bodily fluids. The outer layer may also be configured to dry relatively quickly after receiving the bodily fluids which allows the fluid collection assemblies disclosed herein to be used for prolonged periods of time (e.g., periods of time greater than about 24 hours, such as about 24 hours to about 36 hours, about 30 hours to about 42 hours, or about 36 hours to about 48 hours). The outer layer may at least one of efficiently receive the bodily fluids or remain dry, for example, due to at least one or more of the hydrophilicity of the outer layer or the thickness of the outer layer. The inner layer is able to at least one of quickly or efficiently receive the bodily fluids from the outer layer. The foam or non-woven inner layer also promotes flow of the bodily fluids received thereby towards the fluid outlet and/or the inlet of the conduit.

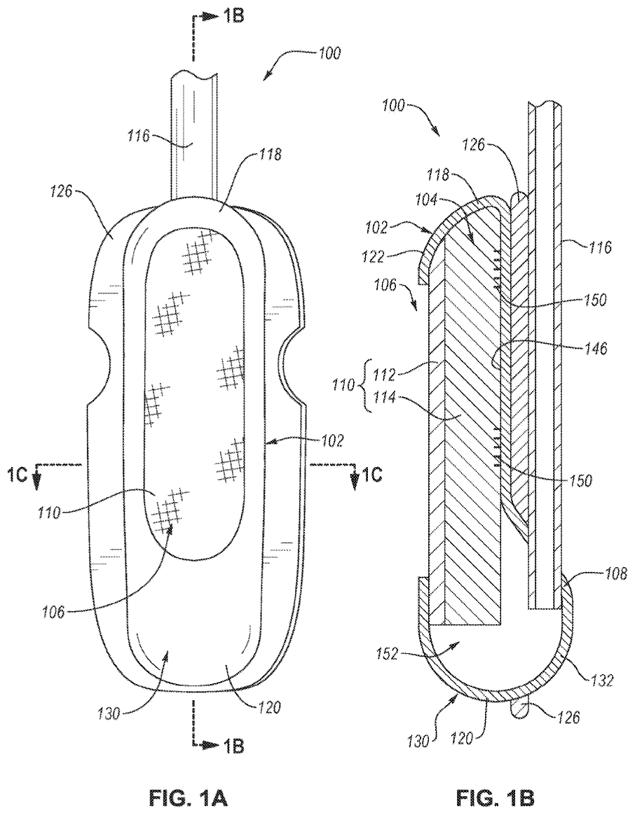

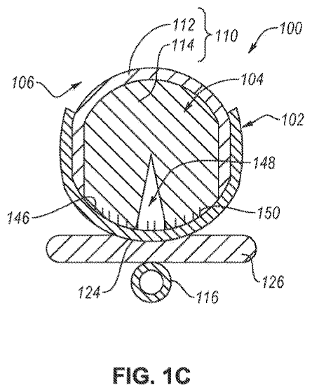

FIG. 1A is an isometric view of a fluid collection assembly 100, according to an embodiment. FIGS. 1B and 1C are cross-sectional schematics of the fluid collection assembly 100 taken along planes 1B-1B and 1C-1C, respectively, according to an embodiment. The fluid collection assembly 100 is example of a female fluid collection assembly for receiving and collecting bodily fluids from a female. The fluid collection assembly 100 includes a fluid impermeable layer 102. The fluid impermeable layer 102 at least defines a chamber 104, at least one opening 106, and a fluid outlet 108. The fluid collection assembly 100 also includes a porous material 110 positioned within the chamber 104. The porous material 110 includes an outer layer 112 (e.g., a fluid permeable membrane) and an inner layer 114 (e.g., a fluid permeable support). The fluid collection assembly 100 may also include at least one conduit 116 partially positioned within the fluid outlet 108 that is configured to remove one or more bodily fluids from the chamber 104. The conduit 116 may not extend through the porous material 110.

The fluid impermeable layer 102 may include a proximal end region 118 and a distal end region 120 opposite the proximal end region 118. Generally, during use, the distal end region 120 is closer to the gluteal cleft of the individual than the proximal end region 118. The fluid impermeable layer 102 may be formed from silicone, neoprene, a thermoplastic elastomer, or other fluid impermeable material.

The opening 106 may be an elongated hole in the fluid impermeable layer 102. For example, the opening 106 may be defined as a cutout in the fluid impermeable layer 102. The opening 106 may be located and shaped to be positioned adjacent to a female urethral opening. The opening 106 may have an elongated shape because the space between the legs of a female is relatively small when the legs of the female are closed, thereby only permitting the flow of the bodily fluids along a path that corresponds to the elongated shape of the opening 106 (e.g., longitudinally extending opening 106).

The fluid collection assembly 100 may be positioned proximate to the female urethral opening and the bodily fluids may enter the chamber 104 of the fluid collection assembly 100 via the opening 106. The fluid collection assembly 100 is configured to receive the bodily fluids into the chamber 104 via the opening 106. When in use, the opening 106 may have an elongated shape that extends from a first location below the urethral opening (e.g., at or near the anus or the vaginal opening) to a second location above the urethral opening (e.g., at or near the top of the vaginal opening or the pubic hair).

In an embodiment, the fluid impermeable layer 102 includes one or more flanges 126. The flanges 126 may provide more locations for underwear or other clothing to contact and press against the fluid collection assembly 100 which may facilitate securing the fluid collection assembly 100 to the vaginal region of the individual and may improve patient comfort. In an embodiment, the flanges 126 may include at least one of an upper flange forming at least a portion of the proximal end region 118 or a bottom flange opposite the upper flange that forms at least a portion of the distal end region 120.

The flanges 126 of the body may extend from the rest of the fluid impermeable layer 102 by a distance that is about 1 mm or greater, about 1 mm or greater, about 3 mm or greater, about 4 mm or greater, about 5 mm or greater, about 6 mm or greater, about 7.5 mm or greater, about 1 cm or greater, about 1.25 cm or greater, about 1.5 cm or greater, about 1 cm or greater, about 1.5 cm or greater, about 3 cm or greater, about 4 cm or greater, about 5 cm or greater, or in ranges of about 1 mm to about 3 mm, about 1 mm to about 4 mm, about 3 mm to about 5 mm, about 4 mm to about 6 mm, about 5 mm to about 7.5 mm, about 6 mm to about 1 cm, about 7.5 mm to about 1.25 cm, about 1 cm to about 1.5 cm, about 1.25 cm to about 1 cm, about 1.5 cm to about 1.5 cm, about 1 cm to about 3 cm, about 1.5 cm to about 4 cm, or about 3 cm to about 5. The distance that the flanges 126 extend from the rest of the fluid impermeable layer 102 may be selected based on the expected size of the vaginal region of the individual (e.g., larger flanges 126 for a larger vaginal region) or the expected rotational forces applied to the fluid collection assembly 100 during use. In some examples, at least some of the flanges 126 may extend further from the rest of the fluid impermeable layer 102 that other flanges 126. For instance, the bottom flange may extend further from the rest of the fluid impermeable layer 102 than the upper flange since some individuals may find the longer bottom flange more comfortable.

In an embodiment, the one or more flanges 126 may exhibit a concave curve relative to the front side of the shell. The concave curve of the flanges 126 may extend from the proximal end region 118 to the distal end region 120. The concave curve of the flanges 126 may allow the flanges 126 to better conform to the shape of the vaginal region since the vaginal region is curved. Conforming the flanges 126 to the shape of the vaginal region may make the fluid collection assembly 100 more comfortable by more uniformly distributing pressure across the vaginal region, especially when the flanges 126 contact the labia majora. In an embodiment, the flanges 126 may be planar. In such an embodiment, the flanges 126 may be compliant thereby allowing the flanges to be curved.

In an embodiment, the fluid impermeable layer 102 may include a sump 130 at or near the distal end region 120. The sump 130 may or may not extend outwardly from the front side 122 of the shell. During use, the sump 130 is configured to be at, near, or otherwise in fluid communication with a gravimetric low point of the porous material 110. For example, the sump 130 may receive a portion of the porous material 110 therein. The sump 130 may receive at least some of the bodily fluids that are received by the porous material 110. The sump 130 may prevent or at least inhibit bodily fluids from leaking from the fluid collection assembly 100.

The sump 130 may extend through the portions of the fluid impermeable layer 102 that form the flanges 126. For example, the sump 130 may include a bulge 132 that extends through a hole defined by the portions of the fluid impermeable layer 102 that form the flanges 126. The bulge 132 of the sump 130 increases the volume of the sump 130 which, in turn, increases the quantity of bodily fluids that may be held within the sump 130. The bulge 132 of the sump 130 also allows the sump 130 to define the fluid outlet 108 when the fluid outlet 108 is located adjacent to the back surface 124 of the fluid impermeable layer 102.

In an embodiment, the fluid impermeable layer 102 exhibits single piece construction (e.g., is integrally formed). In an embodiment, the fluid impermeable layer 102 includes a plurality of pieces attached together to form the complete layer. In an example, as illustrated, the fluid impermeable layer 102 includes a first piece (e.g., a shell) that defines the chamber 104, opening 106, the fluid outlet 108, and the sump 130 and a second piece that forms the flanges 126. In such an embodiment, the first and second pieces may be attached together, for example, using an adhesive or an ultrasonic weld. In an example, the fluid impermeable layer 102 may include a first piece (e.g., a shell) and a second piece (e.g., a connector piece) attached to the first piece. The second piece may extend through the portions of the fluid impermeable layer 102 that form the flanges 126 (e.g., instead of the bulge 132) and form the fluid outlet 108. Further examples of fluid impermeable layers formed from a plurality of pieces are disclosed in PCT Patent Application No. PCT/US2022/032424 filed Jun. 7, 2022 and PCT Patent Application No. PCT/US2022/022111 filed Mar. 28, 2022, the disclosure of each of which is incorporated herein, in its entirety, by this reference.

In an embodiment, the fluid impermeable layer 102 (e.g., the portion of the fluid impermeable layer 102 forming the flanges 126) may define a recess or passageway that is configured to receive a conduit 116. The recess may extend from or near the proximal end region 118 to or near the distal end region 120 thereby allowing the conduit 116 to extend from or near the individual's abdominal region to the fluid outlet 108. In an embodiment, the recess may be configured such that the fluid impermeable layer 102 encloses and/or abuts less than 50% of a circumference of the conduit 116, thereby allowing the conduit 116 to freely enter and leave the recess during use. Allowing the conduit 116 to freely enter and leave the recess may facilitate positioning of the fluid collection assembly 100 such that the porous material 110 is adjacent to the vaginal region even when the conduit 116 is bending away from the vaginal region. Also, allowing the conduit 116 to freely enter and leave the recess may increase the likelihood that movement of the conduit 116 does not move the porous material 110 relative to the vaginal region since movement of the porous material 110 may cause leaking. In an embodiment, at least a portion of the recess may be configured such that the fluid impermeable layer 102 encloses and/or abuts more than 50% (e.g., 51% to about 55%, about 53% to about 57%, or about 55% to about 60%) of the circumference of the conduit 116. Enclosing more than 50% of the circumference of the conduit 116 may more securely attach the conduit 116 to the shell and may allow the conduit 116 to provide additional structure to the shell. The percentage of the conduit 116 enclosed and/or abutted by the shell may be selected such that the inherent elasticity of at least one the shell or the conduit 116 allows the conduit 116 to be easily snapped into and out of the recess. As such, the conduit 116 may be removed from the recess to facilitate positioning the porous material 110 adjacent to the vaginal region or when the conduit 116 is moved.

As previously discussed, the fluid collection assembly 100 includes a porous material 110 disposed in the chamber 104. At least a portion of the porous material 110 may be configured to wick any bodily fluids away from the opening 106, thereby preventing the bodily fluids from escaping the chamber 104. The porous material 110 may also wick the bodily fluids generally towards an interior of the chamber 104. The permeable properties referred to herein may be wicking, capillary action, diffusion, or other similar properties or processes, and are referred to herein as “permeable” and/or “wicking.” Such “wicking” and/or “permeable” properties may not include absorption of the bodily fluids into at least a portion of the porous material 110. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the porous material 110 (e.g., absorbency), such as less than about 30 wt % of the dry weight of the porous material 110, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the porous material 100. In an embodiment, the porous material 110 may include at least one absorbent or adsorbent material.

As previously discussed, the porous material 110 include the outer layer 112 and the inner layer 114. In an embodiment, the porous material 110 only includes the outer layer 112 and the inner layer 114. In an embodiment, the porous material 110 includes one or more additional layers, such as an outermost layer disposed over the outer layer 112. The outermost layer may include, for example, gauze, cotton, or another material that is more comfortable than the outer layer 112.

The outer layer 112 may extend across at least a portion (e.g., all) of the opening 106. The outer layer 112 is configured to quickly receive bodily fluids discharged from the urethral opening. In other words, the outer layer 112 is configured to quickly move the bodily fluids through the opening 106 and into the chamber 104 (e.g., away from the opening 106) thereby preventing or at least inhibiting bodily fluids from leaking from the porous material 110.

The outer layer 112 may be formed from a hydrophilic material. Forming the outer layer 112 from a hydrophilic material is contrary to at least some conventional fluid collection assemblies. For example, continual contact between a porous material containing bodily fluids and a vaginal region (e.g., urethral opening, labia folds, etc.) of an individual may cause at least one of skin degradation (e.g., rashes) or general discomfort. It is generally well known that hydrophilic porous material retain more bodily fluids therein compared to hydrophobic porous materials. In other words, it is conventionally believed that hydrophilic porous material are more likely to cause the vaginal region to remain in continual contact with the bodily fluids compared to a hydrophobic porous material. As such, the porous materials of conventional fluid collection assemblies generally only include hydrophobic materials to prevent or at least minimize bodily fluids stored therein that may contact the vaginal region. Contrary to the general knowledge and such conventional fluid collection assemblies, it has been surprisingly found that the outer layer 112 disclosed herein does not retain bodily fluids therein for the reasons disclosed herein even though the outer layer 112 is formed from a hydrophilic material. Instead, it has been unexpectedly found that the outer layer 112 is more likely to be dry compared the hydrophobic porous material of at least some conventional fluid collection assemblies.

As previously discussed, the outer layer 112 is formed from a hydrophilic material. The hydrophilic material of the outer layer 112 may include a porous material exhibiting a contact angle with water (a major constituent of bodily fluids) that is less than 90°, such as in ranges of about 1° to about 20°, about 10° to about 30°, about 20° to about 40°, about 30° to about 50°, about 40° to about 60°, about 50° to about 70°, about 60° to about 80°, or about 70° to about 89°. Generally, increasing the hydrophilicity of the outer layer 112 (e.g., decreasing the contact angle between the outer layer 112 and water) improves the ability of the outer layer 112 to at least one of quickly or effectively receive bodily fluids. In other words, increasing the hydrophilicity of the outer layer 112 allows the outer layer 112 to receive large quantities of bodily fluids that are discharged over a short period of time. Also, increasing the hydrophilicity of the outer layer 112 generally prevents or at least inhibits bodily fluids from leaking from the porous material 110. However, increasing the hydrophobicity of the outer layer 112 may at least one of limit the materials that may form the outer layer 112 which may increase the cost of forming the outer layer 112 or may inhibit the outer layer 112 from being formed from a comfortable (e.g., soft and smooth) material. As such, the selection of the material that forms the outer layer 112 may be made based on balancing these factors.

In an embodiment, the outer layer 112 may include at least one of bamboo, cellulose, or another natural material. Bamboo and cellulose are cheap, readily available materials that are hydrophobic and comfortable when pressed against the vaginal region. The outer layer 112 including bamboo may also include bamboo kun (a chemical naturally found in bamboo) which may exhibit antimicrobial properties which may allow the outer layer 112 to remain in contact with the vaginal region longer without causing urinary tract infections.

In an embodiment, the outer layer 112 may be formed from synthetic materials, such as at least one of polypropylene, polyethylene, or another suitable synthetic material. Polypropylene and polyethylene are cheap, readily available materials that are generally comfortable when pressed against the vaginal region. However, polypropylene and polyethylene are both natural hydrophobic materials (e.g., exhibit a contact angle with water that is greater than 90°). As such, the outer layer 112 may include at least one of treated polypropylene or treated polyethylene. In an embodiment, the treated polypropylene or treated polyethylene refers to polypropylene or polyethylene that has been immersed in a solution that causes the polypropylene or polyethylene to be hydrophilic. In an embodiment, the treated polypropylene or treated polyethylene refers to polypropylene or polyethylene that has been at least partially coated with a hydrophilic material.

In an embodiment, the outer layer 112 may be formed from a nonwoven material. In an example, the nonwoven material of the outer layer 112 may include at least one carded web which, due the anisotropic structure thereof, allows the strength and flow characteristics of the outer layer 112 to be selected based on the orientation of the fibers thereof. In an example, the outer layer 112 may include at least one needle punched web since needle punched webs exhibits good flow features, especially through the thickness thereof. In an example, the outer layer 112 may include at least one air laid web because air laid webs generally exhibit a high porosity and/or a high loft. In an example, the nonwoven material of the outer layer 112 may include at least one spunbonded web since spunbonded webs generally exhibit a high porosity, a high loft, and/or relatively good water absorption. In an example, the nonwoven material of the outer layer 112 may include at least one spunlaced web since spunlaced webs may more comfortably contact the skin of the patient than at least some other nonwoven materials. In an example, the nonwoven material of the outer layer 112 may include at least one vertical lapped nonwoven fabric, at least one horizontal lapped nonwoven fabric, or at least one cross lapped nonwoven fabric since the nonwoven materials may generally cause the bodily fluids to wick both horizontally and vertically. In an example, the nonwoven material of the outer layer 112 may include any other suitable nonwoven material or combinations of any of the foregoing nonwoven materials. In an embodiment, the outer layer 112 includes a woven material instead of or in addition to a nonwoven material. Forming the outer layer 112 from a woven material may increase the durability of the porous material 110 than if the outer layer 112 is formed from a nonwoven material. However, forming the outer layer 112 from a woven material may decrease the compressibility of the porous material 110 thereby making the porous material 110 less comfortable and/or may make conforming the porous material 110 to the vaginal region more difficult.

The outer layer 112 may be selected to exhibit a density of about 50 kg/m³ to about 100 kg/m³, about 75 kg/m³ to about 125 kg/m³, about 100 kg/m³ to about 150 kg/m³, about 125 kg/m³ to about 175 kg/m³, about 150 kg/m³ to about 200 kg/m³, about 175 kg/m³ to about 225 kg/m³, about 200 kg/m³ to about 250 kg/m³, about 225 kg/m³ to about 275 kg/m³, about 250 kg/m³ to about 300 kg/m³, about 275 kg/m³ to about 325 kg/m³, about 300 kg/m³ to about 350 kg/m³, about 325 kg/m³ to about 375 kg/m³, about 350 kg/m³ to about 400kg/m³, about 375 kg/m³ to about 425 kg/m³, about 400 kg/m³ to about 450 kg/m³, about 425 kg/m³ to about 475 kg/m³, about 450 kg/m³ to about 500 kg/m³, about 475 kg/m³ to about 525 kg/m³, about 500 kg/m³ to about 550kg/m³, about 525 kg/m³ to about 575 kg/m³, or about 550 kg/m³ to about 600 kg/m³.

As previously discussed, the outer layer 112 may be formed from a hydrophilic material which may cause the outer porous material to retain the bodily fluids therein. To decrease the quantity of bodily fluids retained by the outer layer 112, the outer layer 112 may be configured to be relatively thin. For example, the outer layer 112 may be configured to exhibit a thickness measured perpendicularly to the longitudinal axis (e.g., measured radially) that is about 250 μm or less, about 200 μm or less, about 150 μm or less, about 130 μm or less, about 100 μm or less, about 75 μm or less, about 60 μm or less, about 50 μm or less, about 40 μm or less, about 30 μm or less, about 25 μm or less, about 20 μm or less, about 15 μm or less, about 10 μm or less, or in ranges of about 10 μm to about 20 μm, about 15 μm to about 25 μm, about 20 μm to about 30 μm, about 25 μm to about 40 μm, about 30 μm to about 50 μm, about 40 μm to about 60 μm, about 50 μm to about 75 μm, about 60 μm to about 100 μm, about 75 μm to about 130 μm, about 100 μm to about 150 μm, about 130 μm to about 200 μm, or about 150 μm to about 250 μm. The relatively small thickness of the outer layer 112 decreases the overall volume of the outer layer 112 thereby decreasing the volume of bodily fluids that may be retained in the outer layer 112. The decreasing volume of bodily fluids held within the outer layer 112 allows airflow through the chamber 104 to quickly evaporate the bodily fluids that are retained in the outer layer 112, thereby maintaining the outer layer 112 dry. Further, decreasing the thickness of the outer layer 112 may allow the inner layer 114 to pull more bodily fluids from the outer layer 112. It is noted that it has been found that increasing the thickness above about 250 μm may also adversely affect the flow of bodily fluids therethrough.

The outer porous material of the porous material 110 may be selected to exhibit a basis weight of about 10 g/m² to about 20 g/m², about 15 gm/m² to about 25 g/m², about 20 g/m² to about 30 g/m², about 25 g/m² to about 35 g/m², about 30 g/m² to about 40 g/m², about 35 g/m² to about 45 g/m², about 40 g/m² to about 50 g/m², about 45 g/m² to about 55 g/m², about 50 g/m² to about 60 g/m², about 55 g/m² to about 70 g/m², about 60 g/m² to about 80 g/m², about 70 g/m² to about 90 g/m², about 80 g/m² to about 100 g/m², about 90 g/m² to about 110 g/m², or about 100 g/m² to about 120 g/m². The basis weight of the outer layer 112 is a function of the density and thickness thereof. As such, the basis weight of the outer layer 112 may be selected for any of the same reasons as the density and thickness of the outer layer 112.

The outer layer 112 is formed from a plurality of fibers. The plurality of fibers may exhibit an average length and an average lateral dimension (e.g., diameter). In an example, the plurality of fibers may be selected to exhibit an average length that is about 500 μm to about 2 mm, about 1 mm to about 3 mm, about 2 mm to about 4 mm, about 3 mm to about 5 mm, about 4 mm to about 6 mm, about 5 mm to about 7 mm, about 6 mm to about 8 mm, about 7 mm to about 9 mm, about 8 mm to about 1 cm, about 9 mm to about 1.2 cm, about 1 cm to about 1.4 cm, about 1.2 cm to about 1.6 cm, about 1.4 cm to about 1.8 cm, about 1.6 cm to about 2 cm, about 1.8 cm to about 2.25 cm, about 2 cm to about 2.5 cm, about 2.25 cm to about 2.75 cm, about 2.5 cm to about 3 cm, about 2.75 cm to about 3.25 cm, about 3 cm to about 3.5 cm, about 3.25 cm to about 3.75 cm, about 3.5 cm to about 4 cm, about 3.75 cm to about 4.25 cm, about 4 cm to about 4.5 cm, about 4.25 cm to about 4.75 cm, about 4.5 cm to about 5 cm, about 4.75 cm to about 5.5 cm, about 5 cm to about 6 cm, about 5.5 cm to about 6.5 cm, about 6 cm to about 7 cm, about 6.5 cm to about 7.5 cm, about 7 cm to about 8 cm, about 7.5 cm to about 8.5 cm, about 8 cm to about 9 cm, about 8.5 cm to about 9.5 cm, or about 9 cm to about 10 cm. In an example, the fibers may exhibit an average lateral dimension that is about 1 μm to about 2μm, about 1.5 μm to about 3 μm, about 2 μm to about 4 μm, about 3 μm to about 5 μm, about 4 μm to about 7 μm, about 6 μm to about 10 μm, about 8 μm to about 12.5 μm, about 10 μm to about 15 μm, about 12.5 μm to about 17.5 μm, about 15 μm to about 20μm, about 17.5 μm to about 25 μm, about 20 μm to about 30 μm, about 25 μm to about 35 μm, about 30 μm to about 40 μm, about 35 μm to about 45 μm, about 40 μm to about 50 μm, about 45 μm to about 55 μm, about 50 μm to about 60 μm, about 55 μm to about 65 μm, about 60 μm to about 70 μm, about 65 μm to about 75 μm, about 70 μm to about 80 μm, about 75 μm to about 85 μm, about 80 μm to about 90 μm, about 85 μm to about 95 μm, or about 90 μm to about 100 μm. The average length and average lateral dimension of the fibers may be selected such that the fibers exhibit an average aspect ratio. For example, the average length and average lateral dimension of the fibers may be selected such that the fibers exhibit an average aspect ratio (average length: average lateral dimension) of about 100:1 to about 200:1, about 150:1 to about 250:1, about 200:1 to about 300:1, about 250:1 to about 350:1, about 300:1 to about 400:1, about 350:1 to about 450:1, about 400:1 to about 500:1, about 450:1 to about 550:1, about 500:1 to about 600:1, about 550:1 to about 650:1, about 600:1 to about 700:1, about 650:1 to about 750:1, about 700:1 to about 800:1, about 750:1 to about 850:1, about 800:1 to about 900:1, about 850:1 to about 950:1, or about 900:1 to about 1,000:1.

The average length, average lateral dimension, and the average aspect ratio of the fibers may be selected based on a number of factors. In an example, increasing the aspect ratio (e.g., decreasing the average length and/or increasing the average lateral dimension) increases the durability of the outer layer 112 but may decrease the strength of the outer layer 112. In an example, increasing the aspect ratio (e.g., increasing average length) of the fibers may increase the mechanical binding of the fibers. For instance, increasing the aspect ratio of the fibers facilitates entanglement of the fibers which increases the strength and/or durability of the outer porous material. The entanglement of the fibers may also preclude or minimize the amount of other binding techniques that are applied to the outer layer 112, such as heat, chemical binding, or other mechanical binding (e.g., further entanglement caused by needle punching or high-pressure water jets). However, increasing the aspect ratio of the fibers may make dispersion of the fibers more difficult (e.g., uniformity of the outer porous material difficult). Further, increasing the aspect ratio may limit the type of nonwoven webs that may form the outer layer 112. As such, the average length, average lateral dimension, and average aspect ratio of the fibers may be selected based on the desired strength, mechanical binding between the fibers, the amount of processing of the outer porous material (e.g., is further processing to increasing the binding via heat, etc. desired), the type of nonwoven web that includes the fibers, the uniformity of the fibers, etc.

Generally, the average person discharges urine at a rate of about 6 ml/s to about 50 ml/s, such as at a rate of about 10 ml/s to about 25 ml/s. The rate at which the person urinates may vary, such as based on at least one of the size of the person or the age of the person. The outer layer 112 may be selected to receive bodily fluids and have the bodily fluids flow through a portion thereof at a rate that is comparable to the rate at which the individual discharged bodily fluids to prevent leaks. For example, the outer layer 112 may be selected to at least one of receive bodily fluids or have the bodily fluids flow through a portion thereof at a rate that is greater than about 6 ml/s, greater than about 10 ml/s, greater than about 20 ml/s, greater than about 30 ml/s, greater than about 40 ml/s, greater than about 50 ml/s, or in ranges of about 6 ml/s to about 10 ml/s, about 8 ml/s to about 12 ml/s, about 10 ml/s to about 15 ml/s, about 12.5 ml/s to about 17.5 ml/s, about 15 ml/s to about 20 ml/s, about 17.5 ml/s to about 22.5 ml/s, about 20 ml/s to about 25 ml/s, about 22.5 ml/s to about 27.5 ml/s, about 25 ml/s to about 30 ml/s, about 27.5 ml/s to about 35 ml/s, about 30 ml/s to about 40 ml/s, about 35 ml/s to about 45 ml/s, or about 40 ml/s to about 50 ml/s.

The rate at which the outer layer 112 at least one of receives bodily fluids or has the bodily fluids flow through a portion thereof may depend on a number of factors. In an example, the rate at which the outer layer 112 at least one of receives bodily fluids or has the bodily fluids flow through a portion thereof may depend inversely on the density and weight basis of the outer layer 112, wherein increasing the density and/or weight basis of the outer layer 112 may decrease the rate at which the outer layer 112 at least one of receives bodily fluids or has the bodily fluids flow through a portion thereof and vice versa. In an example, the rate at which the outer layer 112 at least one of receives bodily fluids or has the bodily fluids flow through a portion thereof may depend on the hydrophilicity of the outer layer 112. In an example, the rate at which the outer layer 112 at least one of receives bodily fluids or has the bodily fluids flow through a portion thereof may depend on the type of nonwoven web (e.g., carded web, needle punched web, etc.) since each type of nonwoven web may exhibit different rate at which the outer porous material captures and/or transports the bodily fluids.

As previously discussed, the porous material 110 includes an inner layer 114. The inner layer 114 is configured to support the outer layer 112 since the outer layer 112 may be flimsy (e.g., due to the relatively small thickness thereof). For example, the inner layer 114 may be positioned such that the outer layer 112 is disposed between at least a portion of the inner layer 114 and the fluid impermeable layer 102. As such, the inner layer 114 may support and maintain the position of the outer layer 112.

The outer layer 112 may be disposed on an outer surface of the inner layer 114. In an embodiment, the outer layer 112 is positioned on the inner layer 114 to prevent or at least minimize formation of air gaps between the outer layer 112 and the inner layer 114. As used herein, air gaps refers to unoccupied gaps between the outer layer 112 and the inner layer 114 that are significantly larger (e.g., at least 5 times larger or at least 10 times larger) than the average pore size of either of the outer layer 112 and the inner layer 114. In an example, the outer layer 112 is disposed on the inner layer 114 such that at least most 10% (e.g., at most 7.5%, at most 5 %, at more 3 %, at more 2%, or at most 1%) of the surface area of the inner layer 114 adjacent to the outer layer 112 has an air gap adjacent thereto. It has been unexpectedly found that the bodily fluids received by the outer layer 112 flow relatively freely from the outer layer 112 into the inner layer 114. The bodily fluids may flow relatively freely from the outer layer 112 into the inner layer 114 due to hydrogen bonding (e.g., bodily fluids flowing through the inner layer 114 pull bodily fluids from the outer layer 112) or moisture content differentials between the outer layer 112 and the inner layer 114. However, the air gaps between the outer layer 112 and the inner layer 114 form barriers that inhibit flow of the bodily fluids from the outer layer 112 and the inner layer 114. As such, preventing or at least minimizing the formation of air gaps between the outer layer 112 and the inner layer 114 improves flow of the bodily fluids between the outer layer 112 and the inner layer 114.

The inner layer 114 may include any material that may wick, absorb, adsorb, or otherwise allow fluid transport of the bodily fluids, such as any of the fluid outer porous materials disclosed herein above. For example, the outer porous material(s) may be utilized in a more dense or rigid form than in the outer layer 112 when used as the inner layer 114. The inner layer 114 may be formed from any fluid permeable material that is less deformable than the outer layer 112. For example, the inner layer 114 may include a porous polymer (e.g., nylon, polyester, polyurethane, polyethylene, polypropylene, polyvinyl chloride, etc.) structure or an open cell foam. In an example, the inner layer 114 may include spun nylon fiber, a polyurethane foam, a polyethylene foam, or a polyvinyl chloride foam. In some examples, the inner layer 114 may include a nonwoven (e.g., a vertical nonwoven web or any other nonwoven web disclosed herein) or woven material. In some examples, the inner layer 114 may be formed from a natural material, such as cotton, wool, silk, bamboo, or combinations thereof. In such examples, the material may have a coating to prevent or limit absorption of fluid into the material, such as a water repellent coating. In some examples, the inner layer 114 may be formed from fabric, felt, gauze, or combinations thereof.

In an embodiment, at least a portion of the inner layer 114 may be hydrophobic. The inner layer 114 may be hydrophobic when the inner layer 114 exhibits a contact angle with water (a major constituent of bodily fluids) that is greater than about 90°, such as in ranges of about 90° to about 120°, about 105° to about 135°, about 120° to about 150°, about 135° to about 175°, or about 150° to about 180°. The hydrophobicity of the inner layer 114 may limit absorption, adsorption, and solubility of the bodily fluids in the inner layer 114 thereby decreasing the amount of bodily fluids held in the inner layer 114. The lower hydrophilicity of the outer layer 112 may help the porous material 110 receive the bodily fluids from the urethral opening while the hydrophobicity of the inner layer 114 limits the bodily fluids that are retained in the porous material 110.

The inner layer 114 may exhibit a thickness (e.g., radius and/or diameter) that is about 1 mm or greater, about 2 mm or greater, about 4 mm or greater, about 6 mm or greater, about 8 mm or greater, about 10 mm or greater, about 12 mm or greater, about 14 mm or greater, about 16 mm or greater, about 18 mm or greater, about 20 mm or greater, about 22 mm or greater, about 25 mm or greater, or in ranges of about 1 mm to about 4 mm, about 2 mm to about 6 mm about 4 mm to about 8 mm, about 6 mm to about 10 mm, about 8 mm to about 12 mm, about 10 mm to about 14 mm, about 12 mm to about 16 mm, about 14 mm to about 18 mm, about 16 mm to about 20 mm, about 18 mm to about 22 mm, or about 20 mm to about 25 mm. Generally, increasing the thickness of the inner layer 114 increases the quantity of bodily fluids that may at least one of be temporarily stored therein or flow therethrough thereby decreasing the likelihood that the fluid collection assembly 100 leaks. However, increasing the thickness of the inner layer 114 may at least one of dilute any suction force applied to the chamber 104 or make it difficult to position the fluid collection assembly 100 adjacent to a urethral opening.

In an embodiment, the inner layer 114 includes at least one inner porous material. As used herein, the inner porous material includes at least one of a vertical lapped nonwoven material, a polyurethane foam, a polyvinyl chloride foam, or a polyethylene foam. The inner porous material is able to quickly receive bodily fluids from the individual, even when the individual discharges a large quantity of bodily fluids over a short period of time. In an example, the inner porous material may facilitate moving the bodily fluids through the chamber 104 of the fluid collection assembly 100 and towards an outlet (e.g., the fluid outlet 108 or an inlet of a conduit 116 disposed through the fluid outlet 108) which allows the porous material 110 to remain dry. Further, it has been surprisingly found that the bodily fluids received into the outer porous material may flow easily from the outer porous material into the inner porous material and the inner porous material pulls bodily fluids from the outer porous material that would otherwise remain in outer layer 112.

In an embodiment, the inner layer 114 may include a vertical nonwoven material. It has been found that forming the inner layer 114 from the vertical nonwoven material allows the inner layer 114 to quickly remove the bodily fluids from the outer layer 112 and transport the bodily fluids towards the fluid outlet 108 and/or the inlet of the conduit 116. As such, forming the inner layer 114 from a vertical nonwoven material both keeps the porous material 110 more dry compared to conventional fluid collection assemblies. The vertical nonwoven material of the inner layer 114 is formed from a nonwoven web (e.g., any of the nonwoven webs disclosed herein) that is folded. The vertical nonwoven material of the inner layer 114 may include a plurality of folded portions and a plurality of intermediate portions extending between the folded portions. The vertical nonwoven material of the inner layer 114 may include an outer surface (e.g., a surface abutting the outer layer 112) and an opposing inner surface. The folded portions of the vertical nonwoven material of the inner layer 114 may extend generally parallel to the outer and inner surfaces thereof. The intermediate portions may extend between the inner and outer surfaces. In an embodiment, when at least a region of the inner layer 114 exhibits a cylindrical shape, the folded portions of the cylindrical region of the inner layer extend generally parallel to a longitudinal (e.g., central) axis of the porous material 110 and/or extend circumferentially. In an embodiment, when at least a region of the inner layer 114 exhibits a cylindrical shape, the intermediate portions may extend generally parallel to the longitudinal axis of the porous material 110 and/or extend radially relative to the longitudinal axis.

Due to the properties of the vertical nonwoven material, the inner layer 114 may exhibit a narrower thickness than disclosed above when the inner layer includes the vertical nonwoven material. For example, the thickness of the inner layer 114 may be about 8 mm to about 20 mm, such as in ranges of about 8 mm to about 10 mm, about 9 mm to about 11 mm, about 10 mm to about 12 mm, about 11 mm to about 13 mm, about 12 mm to about 14 mm, about 13 mm to about 15 mm, about 14 mm to about 16 mm, about 15 mm to about 17 mm, about 16 mm to about 18 mm, about 17 mm to about 19 mm, or about 18 mm to about 20 mm.

The inner layer 114 includes a vertical nonwoven material, the inner layer 114 may exhibit a density of about 100 kg/m² cm or greater, about 125 kg/m²·cm or greater, about 150 kg/m²·cm or greater, about 175 kg/m²·cm or greater, about 200 kg/m²·cm or greater, or in ranges of about 100 kg/m²·cm to about 150 kg/m²·cm, about 125 kg/m²·cm to about 175 kg/m²·cm, or about 150 kg/m²·cm to about 200 kg/m²·cm. Generally, increasing the density of the vertical nonwoven material increases the strength of the inner layer 114. However, increasing the density of the vertical nonwoven material may at least one of decrease the porosity of the inner layer 114 which decreases the quantity of bodily fluids that may be temporarily stored in the inner layer 114 or decrease the flow rate of the bodily fluids through the inner layer 114. As such, the density of the vertical nonwoven material may be selected based on balancing the desired strength, porosity, and/or flow rate of the bodily fluids through the inner layer 114.

In an embodiment, the inner layer 114 may include a foam. Examples of foams that may form a polyurethane foam, a polyvinyl chloride foam, or a polyethylene foam. It has unexpectedly been found that forming the inner layer 114 from the polyurethane foam, the polyvinyl chloride foam, or the polyethylene foam allows the inner layer 114 to be either hydrophilic or hydrophobic. For example, the polyurethane foam and the polyvinyl chloride foam are natural hydrophilic while the polyethylene foam is naturally hydrophobic thought, it is noted, these foams may be treated to exhibit a hydrophilicity or hydrophobicity that is not natural. It is currently believed that the structure of these foams allows the inner layer 114 to be hydrophilic or hydrophobic while remaining dry. Configuring the inner layer 114 to be hydrophilic promotes flow of the bodily fluids into the inner layer 114 and, unexpectedly, does not cause the inner layer 114 to remain bodily fluids. Configuring the inner layer 114 to be hydrophilic is contrary to at least some conventional fluid collection assemblies. For example, at least some conventional fluid collection assemblies that include an inner layer select the inner layer to be hydrophobic thereby preventing the inner layer from retaining bodily fluids. However, the hydrophobicity of the inner layer of the conventional fluid collection assemblies may inhibit bodily fluids entering the inner layer.

Due to the properties of the polyurethane foam, the polyvinyl chloride foam, and the polyethylene foam, the inner layer 114 may exhibit an average porosity of about 7.5 pores/cm² to about 12.5pores/cm², such as in ranges of about 7.5 pores/cm² to about 8.5pores/cm², about 8 pores/cm² to about 9pores/cm², about 8.5 pores/cm² to about 9.5 pores/cm², about 9 pores/cm² to about 10pores/cm², about 9.5 pores/cm² to about 10.5 pores/cm², about 10 pores/cm² to about 11pores/cm², about 10.5 pores/cm² to about 11.5 pores/cm², about 11 pores/cm² to about 12pores/cm², or about 11.5 pores/cm² to about 12.5pores/cm² . Generally, increasing the number of pores/cm² of the foam increases the number of interconnected pores formed in the porous material, increases the quantity of bodily fluids that may be stored in the foam, and/or the quantity of and rate at which the bodily fluids may flow through the foam. However, increasing the number of pores/cm² decreases the strength of the foam. As such, the number of pores/cm² of the foam may be selected based on balancing these factors.

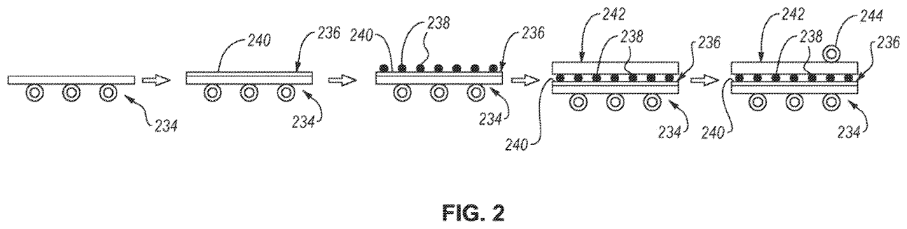

Also, when the inner layer 114 includes at least one of the polyurethane foam, the polyvinyl chloride foam, or the polyethylene foam, the inner layer may exhibit a density of about 15 kg/m²·cm to about 125 kg/m²·cm, such as in ranges of about 15 kg/m²·cm to about 30 kg/m²·cm, about 20 kg/m²·cm to about 40 kg/m²·cm, about 30 kg/m²·cm to about 50 kg/m²·cm, about 40 kg/m²·cm to about 75 kg/m²·cm, about 50 kg/m²·cm to about 100 kg/m²·cm, or about 75 kg/m²·cm to about 125 kg/m²·cm. Generally, increasing the density of the foam increases the strength of the inner layer 114. However, increasing the density of the foam may decrease the porosity of the inner porous material which decreases the quantity of bodily fluids that may be temporarily stored in the porous material 110 and/or decrease the flow rate of the bodily fluids through the inner porous material. As such, the density of the foam may be selected based on balancing the desired strength, porosity, and/or flow rate of the bodily fluids through the inner porous material FIG. 2 is a schematic illustration of a method to form the porous material 110, according to an embodiment. The method illustrated in FIG. 2 uses a conveyor belt 234 to move the components of the porous material from one stage to another stage. However, it is noted that the method of forming the porous material may use devices or structures other than the conveyor belt 234. For example, the method may be performed by one or more people (e.g., single person) at one or more workstations (e.g., a single workstation).

A first sheet 236 may be disposed on the conveyor belt 234. The first sheet 236 is configured to form one of the outer layer 112 or the inner layer 114. In an example, the first sheet 236 may be a sheet including at least one of bamboo fibers, cellulose fiber, treated polypropylene fibers, or polyethylene fibers that form a woven or, more preferably, nonwoven material when the first sheet 236 forms the outer layer 112. In an example, the first sheet 236 may be a sheet including at least one of a vertical nonwoven material, a polyurethane foam, a polyvinyl foam, or a polyethylene foam when the first sheet 236 forms the inner layer 114. As will be discussed in more detail below, a second sheet 242 may be disposed on the first sheet 236, wherein the second sheet 242 includes the other of the outer layer 112 or the inner layer 114 that does not form the first sheet 236.

In an embodiment, the method to form the porous material 110 includes disposing an adhesive 238 on a surface 240 of the first sheet 236. The adhesive 238 may include any adhesive 238 that may attach the first sheet 236 to the second sheet 242 thereby maintaining the structure of the porous material 110. For example, the adhesive 238 may include hot melt adhesive. The adhesive 238 may be sprayed onto the surface 240 or otherwise disposed on the surface 240. In an embodiment, the adhesive 238 is disposed on only a portion of the surface 240 which may prevent the adhesive 238 from blocking the flow of the bodily fluids from the second sheet 242 to the first sheet 236. In an embodiment, the method to form the porous material 110 includes attaching the first and second sheets 236, 242 together without using the adhesive 238. In such an embodiment, the first sheet 236 and the second sheet 242 may be attached together using entanglement between the fibers thereof (e.g., entanglement caused by shooting airbursts or water jets through the sheets). In an embodiment, the method to form the porous material 110 does not include attaching the first and second sheets 236, 242 together. In such an embodiment, contact between the porous material 110 and the fluid impermeable layer 102 when the porous material 110 is disposed in the chamber 104 may maintain the structure of the porous material 110.

The method to form the porous material 110 includes disposing the second sheet 242 adjacent to the surface 240 of the first sheet 236. In an embodiment, the second sheet 242 may be disposed adjacent to the surface 240 after the adhesive 238 is disposed on at least a portion of the surface 240. In an embodiment, the second sheet 242 may be disposed directly on the surface 240, such as when the adhesive 238 is not disposed on the surface 240.

In an embodiment, after disposing the second sheet 242 adjacent to the first sheet 236, the method to form the porous material 110 includes positioning the first and second sheets 236, 242 between adjacent rollers 244. Positioning the first and second sheets 236, 242 between the rollers 244 may cause fiber entanglement between the first and second sheets 236, 242 which, in turn, may help attach the first and second sheets 236, 242 together. In an embodiment, at least one of the rollers 244 may be heated. Positioning the first and second sheets 236, 242 between the heated rollers 244 may cause the adhesive 238 to melt thereby attaching the first and second sheets 236, 242 together.

It is noted that the porous materials disclosed herein may be formed using methods other than the method illustrated in FIG. 2. For example, the outer layer 112 and the inner layer 114 may be coextruded.

Referring back to FIGS. 1A-1C, the as-formed porous material 110 may be disposed in the chamber 104. In an embodiment, as shown in FIG. 1C, the porous material 110 disposed in the chamber 104 may exhibit a generally U-shape. The porous material 110 may exhibit the generally U-shape, for example, when the porous material 110 is initially provided in a sheet-like configuration (similar to the porous material 110 formed according to the method illustrated in FIG. 2). The porous material 110 exhibiting the generally U-shape may be positioned in the chamber 104 such that the outer layer 112 extends across the opening 106 and a surface of the inner layer 114 opposite the outer layer 112 may be positioned adjacent to each other. The porous material 110 exhibiting the U-shape may exhibit several benefits over porous materials exhibiting other shapes. In an example, the outer layer 112 may not be positioned adjacent to a back interior surface 146 of the fluid impermeable layer 102 (i.e., a surface of the fluid impermeable layer 102 opposite the opening 106) when the porous material 110 exhibits the U-shape. The outer layer 112 adjacent to the back interior surface 146 is likely to retain bodily fluids therein due to the hydrophilicity thereof and/or the lack of airflow therein which, in turn, may impede flow of the bodily fluids through the chamber 104. Not positioning the outer layer 112 adjacent to the back interior surface 146 prevents the outer layer 112 from impeding the flow of bodily fluids that are adjacent to the back interior surface 146. In other words, not positioning the outer layer 112 adjacent to the back interior surface 146 improves flow of the bodily fluids through the porous material 110.

In an embodiment, the porous material 110 may define a gap 148 when the porous material 110 exhibits the U-shape. The gap 148 may be defined by the fluid impermeable layer 102 and the inner layer 114. The gap 148 may be a substantially unoccupied space in the chamber 104. The gap 148 may allow the chamber 104 to receive a larger quantity of bodily fluids than if the chamber 104 did not include the gap 148. For example, any bodily fluids that enter the gap 148 may flow quicker towards the fluid outlet 108 and/or the inlet of the conduit 116 than the bodily fluids in the porous material 110. Since the gap 148 is spaced from the opening 106, the fact that the gap 148 is unoccupied does not increase the likelihood that the bodily fluids leak from the chamber 104.

It is noted that the porous material 110 may exhibit a shape other than the U-shape. For example, the porous material 110 may exhibit a generally cylindrical shape where the inner layer 114 is concentrically positioned within the outer layer 112.

The fluid impermeable layer 102 may have difficulty maintaining the porous material 110 is the chamber 104, especially when the porous material 110 exhibits a U-shape. As such, in an embodiment, the fluid impermeable layer 102 may include one or more barbs 150 extending into the chamber 104. The barbs 150 are configured to grab and hold onto the porous material 110 thereby inhibiting the porous material 110 from leaving the chamber 104. For example, the barbs 150 may include plastic hooks similar to the hooks of a hook-and-loop fastener (e.g., Velcro™) since such hooks may grab and hold onto the porous material 110. In an embodiment, the barbs 150 may be located on at least a portion the back interior surface 146 of the fluid impermeable layer 102. The barbs 150 on the back interior surface 146 are able to grip the edges of the porous material 110 when the porous material 110 exhibits the U-shape.

The porous material 110 may at least substantially completely fill the portions of the chamber 104 that are not occupied by the conduit 116. In some examples, the porous material 110 may not substantially completely fill the portions of the chamber 104 that are not occupied by the conduit 116. In such an example, the fluid collection assembly 100 includes the reservoir 152 disposed in the chamber 104.

The reservoir 152 is a substantially unoccupied portion of the chamber 104. The reservoir 152 may be defined between the fluid impermeable layer 102 and one or both of the outer layer 112 and the inner layer 114. The bodily fluids that are in the chamber 104 may flow through the porous material 110 to the reservoir 152. The reservoir 152 may retain of the bodily fluids therein.

The bodily fluids that are in the chamber 104 may flow through the outer layer 112 and/or the inner layer 114 to the reservoir 152. The fluid impermeable layer 102 may retain the bodily fluids in the reservoir 152. While depicted in the distal end region 120 (e.g., within the sump 130), the reservoir 152 may be located in any portion of the chamber 104 such as the proximal end region 118. The reservoir 152 may be located in a portion of the chamber 104 that is designed to be located in a gravimetrically low point of the fluid collection assembly 100 when the fluid collection assembly 100 is worn.

In some examples (not shown), the fluid collection assembly 100 may include multiple reservoirs, such as a first reservoir that is located at the portion of the chamber 104 closest to the inlet of the conduit 116 (e.g., distal end region 120) and a second reservoir that is located at the portion of the of the chamber 104 that is at or near proximal end region 118). In another example, the inner layer 114 is spaced from at least a portion of the conduit 116, and the reservoir 152 may be the space between the inner layer 114 and the conduit 116.

The conduit 116 may be at least partially disposed in the chamber 104. The conduit 116 may be used to remove the bodily fluids from the chamber 104. The conduit 116 includes at least one wall defining an inlet, an outlet (not shown) downstream from the inlet, and a passageway. The outlet of the conduit 116 may be operably coupled to a vacuum source, such as a vacuum pump for withdrawing fluid from the chamber 104 through the conduit 116.

Locating the inlet of the conduit 116 at or near a location expected to be the gravimetrically low point of the chamber 104 when worn by an individual enables the conduit 116 to receive more of the bodily fluids than if inlet of the conduit 116 was located elsewhere and reduce the likelihood of pooling (e.g., pooling of the bodily fluids may cause microbe growth and foul odors). For instance, the bodily fluids in the porous material 110 may flow in any direction due to capillary forces. However, the bodily fluids may exhibit a preference to flow in the direction of gravity, especially when at least a portion of the porous material 110 is saturated with the bodily fluids. Accordingly, one or more of the inlet of the conduit 116 or the reservoir 152 may be located in the fluid collection assembly 100 in a position expected to be the gravimetrically low point in the fluid collection assembly 100 when worn by an individual, such as the distal end region 120.

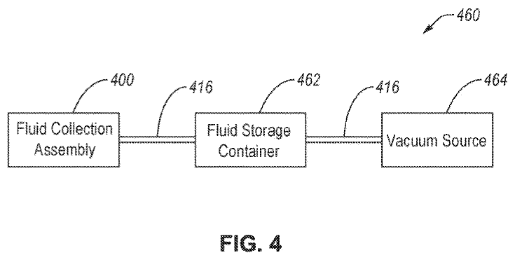

The inlet and the outlet of the conduit 116 are configured to fluidly couple (e.g., directly or indirectly) the vacuum source (not shown) to the chamber 104 (e.g., the reservoir 152). As the vacuum source (464 of FIG. 4) applies a vacuum/suction in the conduit 116, the bodily fluids in the chamber 104 (e.g., at the distal end region 120 such as in the reservoir 152) may be drawn into the inlet of the conduit 116 and out of the fluid collection assembly 100 via the conduit 116. In some examples, the conduit 116 may be frosted or opaque (e.g., black) to obscure visibility of the bodily fluids therein.

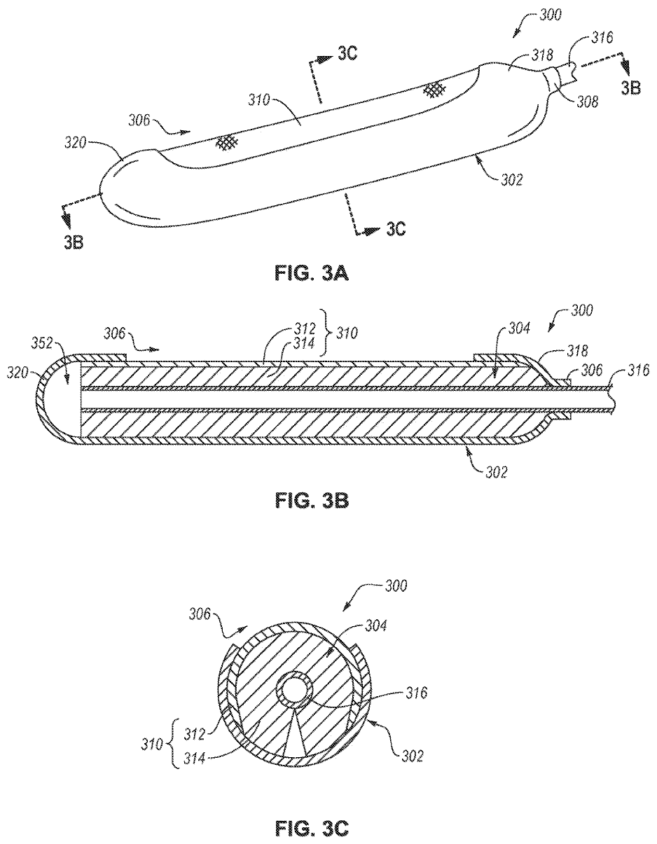

The porous material 110 disclosed herein is especially effective when used with the fluid collection assembly 100. However, it is noted that the porous material 110 (or substantially similar porous materials exhibiting a similar structure and/or materials) may be used with other fluid collection assemblies. For example, FIG. 3A is an isometric view of a fluid collection assembly 300, according to an embodiment. FIGS. 3B and 3C are cross-sectional schematics of the fluid collection assembly 300 taken along planes 3B-3B and 3C-3C, respectively, according to an embodiment. Except as otherwise disclosed herein, the fluid collection assembly 300 is the same as or substantially similar to any of the fluid collection assemblies disclosed herein. For example, the fluid collection assembly 300 may include a fluid impermeable layer 302 defining at least a chamber 304, at least one opening 306, and a fluid outlet 308. The fluid collection assembly 300 also includes a porous material 310 disposed in the chamber 304. The porous material 310 includes an outer layer 312 and an inner layer 314.

In some examples, the fluid impermeable layer 302 may be tubular (ignoring the opening 306), such as substantially cylindrical (as shown), oblong, prismatic, or flattened tubes. During use, the outer surface of the fluid impermeable layer 302 may contact the individual. The fluid impermeable layer 302 may be sized and shaped to fit between the labia and/or the gluteal cleft between the legs of a female user.

In some examples, the fluid impermeable layer 302 may define a fluid outlet 308. The fluid outlet 308 may be located on the proximal end region 318 of the fluid impermeable layer 302. The fluid outlet 308 may be sized to receive the conduit 316. The at least one conduit 316 may be disposed in the chamber 304 via the fluid outlet 308. For example, the conduit 316 may extend into the fluid impermeable layer 302 from the proximal end region 318 and may extend to the distal end region 320 to a point proximate to the reservoir 352 therein such that the inlet of the conduit 316 is in fluid communication with the reservoir 352. The conduit 316 fluidly couples the chamber 304 with the fluid storage container (not shown) or the vacuum source (not shown).

The conduit 316 may extend through a bore in the porous material 310. In an embodiment, the conduit 316 extends from the fluid outlet 308, through the bore, to a location that is proximate to the reservoir 352. In such an embodiment, the inlet of the conduit 316 may not extend into the reservoir 352 and, instead, the inlet of the conduit 316 may be disposed within the porous material 310 or at a terminal end thereof. For example, an end of the conduit 316 may be coextensive with or recessed within the outer layer 312 and/or the inner layer 314. In an embodiment, the conduit 316 is at least partially disposed in the reservoir 352 and the inlet of the conduit 316 may be extended into or be positioned in the reservoir 352. The bodily fluids collected in the fluid collection assembly 300 may be removed from the chamber 304 via the conduit 316.

Further examples of fluid collection assemblies that may include the porous materials disclosed herein are disclosed in U.S. Pat. No. 10,973,678 filed on Jun. 2, 2017, U.S. Pat. No. 10,390,989 filed on Sep. 8, 2016, U.S. Pat. No. 10,226,376 filed on Jun. 3, 2017, PCT Patent Application No. PCT/US2021/039866 filed on Jun. 30, 2021, and U.S. patent application Ser. No. 16/433,773 filed on Jun. 6, 2019, the disclosure of each of which is disclosed herein, in its entirety, by this reference.

FIG. 4 is a block diagram of a fluid collection system 460 for fluid collection, according to an embodiment. The fluid collection system 460 includes a fluid collection assembly 400, a fluid storage container 462, and a vacuum source 464. The fluid collection assembly 400 may be the same or substantially similar to any of the fluid collection assemblies disclosed herein. The fluid collection assembly 400, the fluid storage container 462, and the vacuum source 464 may be fluidly coupled to each other via one or more conduits 416. For example, fluid collection assembly 400 may be operably coupled to one or more of the fluid storage container 462 or the vacuum source 464 via the conduit 416. The bodily fluids collected in the fluid collection assembly 400 may be removed from the fluid collection assembly 400 via the conduit 416 which protrudes into the fluid collection assembly 400. For example, an inlet of the conduit 416 may extend into the fluid collection assembly 400, such as to a reservoir therein. The outlet of the conduit 416 may extend into the fluid collection assembly 400 or the vacuum source 464. Suction force may be introduced into the chamber of the fluid collection assembly 400 via the inlet of the conduit 416 responsive to suction (e.g., vacuum) force applied at the outlet of the conduit 416.

The suction force may be applied to the outlet of the conduit 416 by the vacuum source 464 either directly or indirectly. The suction force may be applied indirectly via the fluid storage container 462. For example, the outlet of the conduit 416 may be disposed within the fluid storage container 462 and an additional conduit 416 may extend from the fluid storage container 462 to the vacuum source 464. Accordingly, the vacuum source 464 may apply suction to the fluid collection assembly 400 via the fluid storage container 462. The suction force may be applied directly via the vacuum source 464. For example, the outlet of the conduit 416 may be disposed within the vacuum source 464. An additional conduit 416 may extend from the vacuum source 464 to a point outside of the fluid collection assembly 400, such as to the fluid storage container 462. In such examples, the vacuum source 464 may be disposed between the fluid collection assembly 400 and the fluid storage container 462.

The fluid storage container 462 is sized and shaped to retain bodily fluids therein. The fluid storage container 462 may include a bag (e.g., drainage bag), a bottle or cup (e.g., collection jar), or any other enclosed container for storing bodily fluids such as urine. In some examples, the conduit 416 may extend from the fluid collection assembly 400 and attach to the fluid storage container 462 at a first point therein. An additional conduit 416 may attach to the fluid storage container 462 at a second point thereon and may extend and attach to the vacuum source 464. Accordingly, a vacuum (e.g., suction) may be drawn through fluid collection assembly 400 via the fluid storage container 462. Bodily fluids, such as urine, may be drained from the fluid collection assembly 400 using the vacuum source 464.

The vacuum source 464 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 464 may provide a vacuum or suction to remove bodily fluids from the fluid collection assembly 400. In some examples, the vacuum source 464 may be powered by one or more of a power cord (e.g., connected to a power socket), one or more batteries, or even manual power (e.g., a hand operated vacuum pump). In some examples, the vacuum source 464 may be sized and shaped to fit outside of, on, or within the fluid collection assembly 400. For example, the vacuum source 464 may include one or more miniaturized pumps or one or more micro pumps. The vacuum sources 464 disclosed herein may include one or more of a switch, a button, a plug, a remote, or any other device suitable to activate the vacuum source 464.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or +2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A fluid collection assembly, comprising:
a fluid impermeable layer defining at least a chamber, at least one opening, and a fluid outlet;
a porous material at least partially disposed in the chamber, the porous material including:
a hydrophilic fluid permeable outer layer including at least one of bamboo, treated polypropylene, or treated polyethylene; and
a fluid permeable inner layer including at least one of a non-woven material or a foam.

2. The fluid collection assembly of claim 1, wherein the porous material only includes a single hydrophilic fluid permeable outer layer and a single fluid permeable inner layer.

3. The fluid collection assembly of claim 1, wherein the porous material exhibits a generally U-shape.

4. The fluid collection assembly of claim 1, wherein the hydrophilic fluid permeable outer layer includes bamboo of

5. The fluid collection assembly of claim 1, wherein the hydrophilic fluid permeable outer layer includes at least one of a treated polypropylene non-woven material or the treated polyethylene.

6. The fluid collection assembly of claim 1, wherein the hydrophilic fluid permeable outer layer exhibits a surface density of about 25 g/m² to about 55 g/m².

7. The fluid collection assembly of claim 1, wherein the hydrophilic fluid permeable outer layer exhibits a thickness of about 25 μm to about 125 μm.

8. The fluid collection assembly of claim 1, wherein the fluid permeable inner layer includes a nonwoven material.

9. The fluid collection assembly of claim 8, wherein the fluid permeable inner layer includes a vertical non-woven material.

10. The fluid collection assembly of claim 9, wherein the vertical non-woven material exhibits a density range of about 100 kg/m²cm to about 200 kg/m²cm.

11. The fluid collection assembly of claim 9, wherein the vertical non-woven material exhibits a thickness of about 8 mm to about 20 mm.

12. The fluid collection assembly of claim 1, wherein the fluid permeable inner layer includes at least one foam.

13. The fluid collection assembly of claim 12, wherein the at least one foam includes at least one of polyurethane foam, a polyvinyl chloride foam, or a polyethylene foam.

14. The fluid collection assembly of claim 12, wherein the at least one foam exhibits an average pore size of about 7.75 pores/cm² to about 12.5 cm².

15. The fluid collection assembly of claim 12, wherein the at least one foam exhibits a density of about 15 kg/m³ to about 125 kg/m³.

16. The fluid collection assembly of claim 1, wherein the porous material further includes an adhesive between the hydrophilic fluid permeable outer layer and the fluid permeable inner layer.

17. The fluid collection assembly of claim 1, further comprising one or more barbs extending from at least one interior back surface of the fluid impermeable layer, wherein the at least one interior back surface defines a portion of the chamber.

18. A fluid collection system, comprising:
the fluid collection assembly of claim 1;
a fluid storage container; and
a vacuum source;
wherein the chamber of the fluid collection assembly, the fluid storage container, and the vacuum source are in fluid communication with each other that, when one or more bodily fluids are present in the chamber, a suction provided from the vacuum source to the chamber of the fluid collection assembly removes the one or more bodily fluids from the chamber and deposits the bodily fluids in the fluid storage container.

19. A method to collect bodily fluids, the method comprising:
positioning at least one opening of a fluid collection assembly adjacent to a female urethral opening, the fluid collection assembly including:
a fluid impermeable layer defining at least a chamber, the at least one opening, and a fluid outlet;
a porous material at least partially disposed in the chamber, the porous material including:
a hydrophilic fluid permeable outer layer including at least one of bamboo, treated polypropylene, or treated polyethylene; and
a fluid permeable inner layer including at least one of a foam or a non-woven material; and
receiving bodily fluids from the female urethral opening into the chamber.

20. A method to form a fluid collection assembly, the method comprising:
providing a porous material, the porous material including a hydrophilic fluid permeable outer layer and a fluid permeable inner layer, the hydrophilic fluid permeable outer layer including at least one of bamboo, treated polypropylene, or treated polyethylene, the fluid permeable inner layer including at least one of a nonwoven material or a foam; and
disposing the porous material through at least one opening and into a chamber, the at least one opening and the chamber defined by a fluid impermeable layer, the fluid impermeable layer defining a fluid inlet.

21. The method of claim 20, wherein providing a porous material includes:
providing a first sheet, the first sheet including one of the hydrophilic fluid permeable outer layer or the fluid permeable inner layer;
after providing the first sheet, disposing an adhesive on at least a portion of a top surface of the first sheet; and
after disposing an adhesive on at least a portion of the top surface of the first sheet, disposing a second sheet on at least the adhesive, the second sheet including the other of the hydrophilic fluid permeable outer layer or the fluid impermeable inner layer.
